(19) United States
(12) Patent Application Publication
(10) Pub. No.: US 2016/0263040 A1
(43) Pub. Date: Sep. 15, 2016

(54) COMPOSITE FORMULATION COMPRISING TADALAFIL AND AMLODIPINE

(71) Applicant: HANMI PHARM. CO., LTD., Hwaseong-si, Gyeonggie-do (KR)

(72) Inventors: Jin Cheul KIM, Seoul (KR); Jae Ho KIM, Gyeonggi-do (KR); Caleb Hyungmin PARK, Gyeonggi-do (KR); Yong Il KIM, Gyeonggi-do (KR); Jae Hyun PARK, Gyeonggi-do (KR); Jong Soo WOO, Gyeonggi-do (KR)

(21) Appl. No.: 15/034,269

(22) PCT Filed: Nov. 6, 2014
(86) PCT No.: PCT/KR2014/010641

§ 371 Date: May 4, 2016

(30) Foreign Application Priority Data
Nov. 15, 2013 (KR) 10-2013-0139334
Jul. 28, 2014 (KR) 10-2014-0096022

(51) Int. Cl. A61K 9/20; A61K 31/4422; A61K 9/16; A61K 31/4985

(52) U.S. Cl. CPC A61K 9/2077; A61K 31/4985; A61K 31/4422; A61K 9/1617; A61K 9/1652; A61K 9/1623; A61K 9/2095; A61K 9/1682; A61K 9/2013; A61K 9/2018; A61K 9/2054; A61K 9/2059

(57) ABSTRACT

A composite formulation is provided which includes: tadalafil or a pharmaceutically acceptable salt thereof as an active ingredient; and amlodipine or a pharmaceutically acceptable salt thereof as an active ingredient, wherein a total amount of tadalafil and amlodipine is in a range of about 6 parts to about 16 parts by weight based on 100 parts by weight of a total weight of the composite formulation.

### TECHNICAL FIELD

The present disclosure relates to a composite formulation including tadalafil and amlodipine, and more particularly, to a composite formulation including both tadalafil and amlodipine as active ingredients each at a sufficient unit dose as used in a conventional single formulation of tadalafil or amlodipine and having a size that is not larger than the conventional single formulation of tadalafil or amlodipine, and thus, having improved patient's compliance.

### BACKGROUND ART

Tadalafil is a selective and reversible cyclic guanosine monophosphate (cGMP)-specific phosphodiesterase type 5 (PDE-5) inhibitor. The inhibition of PDE-5 by tadalafil upon a local release of nitrogen oxide (NOₓ) by sexual stimulation may increase a cGMP level in the corpus cavernosum, cause relaxation of smooth muscle tissue and blood flow into the penis, and consequently, erection. A tdalafil, which is currently sold under the trade name of “Cialis®” tablets, is known to be useful for the treatment of patients with either or both erectile dysfunction and benign prostatic hyperplasia (WO2000/066099).

Hypertension is a main risk factor of cardiovascular diseases such as strokes, myocardial infarction, congestive heart failure, kidney diseases, or peripheral vascular diseases. The risk of cardiovascular diseases may become linearly higher with a blood pressure increase. Blood pressure is more closely related with strokes than with coronary artery diseases. Accordingly, steady blood pressure control is important and long-term use of blood pressure medications is required. In this regard, a deliberate selection of treatment drugs for long-term use is required. Antihypertensive agents may be classified into diuretics, beta-blockers, alpha-beta-blockers, calcium channel antagonists, angiotensin converting enzyme inhibitors, angiotensin receptor blockers, and the like.

Amlodipine (3-ethyl 5-methyl 2-[(2-aminoethoxy)methyl]-4-(2-chlorophenyl)-6-methyl-1,4-dihydropyridine-3,5-dicarboxylate), which is currently sold under the product name of “Norvasc®” by Pfizer, is a calcium channel antagonist, and it is effective lower systolic blood pressure and prevent strokes. Calcium channel antagonists such as amlodipine are effective in angina pectoris due to their coronary artery expansion action, and in particularly, in the treatment of variant angina pectoris with coronary spasms.

According to data from the health insurance in the United States, about 41% of erectile dysfunction patients are known to suffer from hypertension. According to the 2008 Korean National Health and Nutrition Examination Survey by the Korea Centers for Disease Control and Prevention, about 54.3% of hypertension patients are publicly disclosed to have erectile dysfunction. According to the 2009 erectile dysfunction treatment guideline by the British society for sexual medicines, erectile dysfunction and hypertension are related to each other, and either one of them could be a presymptom of the other. The 2013 treatment guideline by the Korean Society of Hypertension clarifies that an angiotensin-conversion enzyme (ACE) inhibitor, an angiotensin blocker, a beta-blocker, a calcium channel antagonist, or a diuretic may be used as primary hypertension medicines, that hypertension patients with erectile dysfunction are required to be intensively asked about their conditions so that their compliance is not affected by erectile dysfunction, and that PDE-5 inhibitors may be administered in combination with hypertension drugs.

Sildenafil, one of the commercially available PDE-5 inhibitors, is sold under the product name “Viagra®” by Pfizer. This drug may be administered on-demand, and its safety in a patient who daily takes blood pressure medications has not been proven yet. However, tadalafil (Cialis®) may be co-administered once a day at a daily unit dose of about 5 mg, together with blood pressure medications. According to the research into co-administration of tadalafil and a calcium channel antagonist, it is reported that a vasodilation function of tadalafil may enhance an antihypertensive function of the calcium channel antagonist, with a low risk of hypotension.

In conclusion, a combination of tadalafil and amlodipine as two active ingredients may be used to treat the accompanied disease, and may prevent or treat the two diseases at the same time. However, it may be inconvenient or not easy for a patient with diseases to take or swallow two different single formulations at the same time, thus causing a low patient's compliance.

### SUMMARY OF INVENTION

### Technical Problem

The present invention provides a composite formulation including both tadalafil and amlodipine as active ingredients each at the same unit dose as used in a conventional single formulation of tadalafil or amlodipine and having a size that is not larger than the conventional single formulation of tadalafil or amlodipine.

The present invention provides a composite formulation including both tadalafil and amlodipine as active ingredients each at the same unit dose as used in a conventional single formulation of tadalafil or amlodipine and having a size that is not larger than the conventional single formulation of tadalafil or amlodipine, without a reduction in a dissolution rate of the active ingredients, compared to the conventional single formulation of tadalafil or amlodipine, and with ensured stability in appearance. The present invention provides methods of preparing the composite formulations.

### Technical Solution

According to an aspect of the present invention, there is provided a composite formulation including: tadalafil or a pharmaceutically acceptable salt thereof and amlodipine or a pharmaceutically acceptable salt thereof as active ingredients, wherein a total amount of tadalafil and amlodipine is in a range of about 6 parts to about 16 parts by weight based on 100 parts by weight of a total weight of the composite formulation. A composite formulation may include: a tadalafil wet granules-part including tadalafil or a pharmaceutically acceptable salt thereof; and an amlodipine mixture-part including amlodipine or a pharmaceutically acceptable salt thereof, wherein a total amount of tadalafil and amlodipine is in a range of about 6 parts to about 16 parts by weight based on 100 parts by weight of a total weight of the composite formulation.

According to an aspect of the present invention, there is provided a method of preparing any of the above-described composite formulations, the method including: preparing a tadalafil wet granules-part including tadalafil or a pharmaceutically acceptable salt thereof, and a pharmaceutically acceptable additive; preparing an amlodipine mixture-part including amlodipine or a pharmaceutically acceptable salt thereof, and a pharmaceutically acceptable additive; and formulating the tadalafil wet granules-part and the amlodipine mixture-part together.

### Advantageous Effects

As described above, a composite formulation may include two active ingredients in a single formulation, may have a size that is not larger than a single formulation of either one of the two active ingredients, and thus may improve patient's compliance who needs to take the two active ingredients. In particular, the composite formulation may allow a patient who has to take daily both tadalafil and amlodipine to easily swallow or take the two active ingredients in a single formulation, thus markedly improving a patient's compliance. Therefore, due to a vasodilation effect of tadalafil as a PDE-5 inhibitor and a antihypertensive function of amlodipine, the composite formulation may be useful for the prevention and treatment of hypertension and cardiovascular diseases accompanied thereby, and for a patient with both hypertension and an erectile dysfunction disease.

A composite formulation according to any of the above-embodiments of the present disclosure may be prepared at low costs by using a preparation method and equipment conventionally available in the pharmaceutical field.

### DESCRIPTION OF DRAWINGS

FIG. 1 is an image comparatively illustrating the sizes of composite formulations of Examples 1 and 2, a composite formulation of Comparative Example 1, and commercially available single formulations Norvasc® and Cialis® as tablets in unit dosage form;

FIG. 2 is a graph of tadalafil dissolution rate with respect to time in composite formulations of Examples 1, 3, and 4, composite formulations of Preparation Examples 1 and 2, and commercially available single formulation Cialis®, obtained using the USP37 dissolution test for tadalafil tablets;

FIG. 3 is a graph of amlodipine dissolution rate with respect to time in the composite formulation of Example 1, the composite formulation of Preparation Example 3, and commercially available single formulation Norvasc®, obtained using the USP37 dissolution test for amlodipine tablets; and

FIG. 4 is a graph of tadalafil dissolution rate with respect time in the composite formulations of Examples 1 and 5, composite formulations of Preparation Examples 4 and 5, and commercially available single formulation Cialis®, obtained using the USP37 dissolution test for tadalafil tablets.

### MODE FOR INVENTION

Unless otherwise defined, all terms (including technical and scientific terms) used herein have the same meaning as commonly understood by one of ordinary skill in the art to which this invention belongs. Although exemplary methods or materials are listed herein, other similar or equivalent ones are also within the scope of the present invention. All publications disclosed as references herein are incorporated in their entirety by reference.

The present inventors formulated a composite formulation including both tadalafil and amlodipine as active ingredients in the single formulation to improve a patient's compliance, wherein the patient has to take the two active ingredients. Preparing a composite formulation, including total ingredients of each unit dosage form of the two individual single formulations, may be easy and may also be advantageous in terms of a patient's compliance, which lead to form a composite formulation with a mass or size corresponding to the sum of each unit dosage form. However, the larger size of the composite formulation, compared to the individual single formulations, may lower the patient's compliance with the composite formulation, and thus it may be meaningless to prepare the composite formulation.

To address this drawback, the present inventors researched to prepare a composite formulation including tadalafil and amlodipine as two active ingredients to have a size no larger than the size of each conventional individual single formulation of tadalafil or amlodipine in order to improve a patient's compliance, which leads to develop a tablet with an increased ratio of the active ingredients to a total weight of the tablet.

According to an aspect of the present disclosure, a composite formulation includes: tadalafil or a pharmaceutically acceptable salt thereof and amlodipine or a pharmaceutically acceptable salt thereof as active ingredients, wherein a total amount of tadalafil and amlodipine is in a range of about 6 parts to about 16 parts by weight based on 100 parts by weight of a total weight of the composite formulation.

The total amount of tadalafil and amlodipine in the composite formulation may be from about 6 parts to about 16 parts by weight bated on 100 parts by weight of the total weight of the composite formulation, indicating that a proportion of the two active ingredients in the composite formulation is higher than conventional single formulations. At the same time, the size of the composite formulation may be no larger than the size of conventional single formulations. Therefore, the composite formulation may allow a patient to take both tadalafil and amlodipine as a single formulation at the same time, may be small enough for smooth swallowing, thus improving a patient's compliance.

When a composite formulation includes a relatively high proportion of active component to a total weight of the composite formulation, this may inevitably result in a low dissolution rate in the aspect of the pharmaceutics. In order for the composite formulation to have the same efficacy and effect as each of conventional single formulations of tadalafil or amlodipine, an equivalent or higher dissolution rate (i.e., about 70% or more in 15 minutes) to the dissolution rate of each conventional single formulation is required. To this end, the inventors of the present invention developed a composite formulation that may overcome a dissolution rate reduction with an increase in active ingredient proportion.

According to an embodiment of the present disclosure, a composite formulation includes: tadalafil or a pharmaceutically acceptable salt thereof and amlodipine or a pharmaceutically acceptable salt thereof as active ingredients, wherein a total amount of tadalafil and amlodipine is in a range of about 6 parts to about 16 parts by weight based on 100 parts by weight of a total weight of the composite formulation, and tadalafil and amlodipine in the composite formulation each have a dissolution rate of about 70% or more in 15 minutes, as tested by the United States Pharmacopeia (USP) 37 dissolution test.

As used herein, the USP37 dissolution test refers to a dissolution test in General Tests and Assays according to the USP37. The dissolution test herein is performed by the USP37 dissolution test for tadalafil tablets or amlodipine tablets.

In some embodiments, the composite formulation may include: a tadalafil wet granules-part including tadalafil or a pharmaceutically acceptable salt thereof; and an amlodipine mixture-part including amlodipine or a pharmaceutically acceptable salt thereof, wherein a total amount of tadalafil and amlodipine may be in a range of about 6 parts to about 16 parts by weight based on 100 parts by weight of a total weight of the composite formulation.

As used herein, the terms “wet granules-part” refers to granules of a mixture prepared by wet granulation of a mixture, and the terms “mixture-part” refers to a mixture with a non-granular form.

In some embodiments, when the composite formulation is prepared to include tadalafil or a pharmaceutically acceptable salt thereof in the wet granules-part, and amlodipine or pharmaceutically acceptable salt thereof in the mixture-part, the dissolution rate of each of tadalafil and amlodipine may be about 70% or more in 15 minutes, as tested by the USP 37 dissolution test. However, when a composite formulation was prepared to include both amlodipine and tadalafil in a wet granule-part, a dissolution rate of amlodipine was found to be significantly low as about 60% or less in 15 minutes, as tested by the USP37 dissolution test (refer to Experimental Example 3).

The tadalafil wet granules-part or the amlodipine mixture-part may include at least one pharmaceutically acceptable additive selected from the group consisting of a diluent, a disintegrant, a binder, and a glidant. The excipient, binder, disintegrant, and glidant may be any common additives known in the art. The amount of the diluent may be in a range of about 20 parts to about 60 parts by weight based on 100 parts by weight of the total weight of the composite formulation. The amount of the binder may be in a range of about 1 part to about 10 parts by weight, and in some embodiments, about 2 parts to about 6 parts by weight, based on 100 parts by weight of the total weight of the composite formulation. The amount of the disintegrant may be in a range of about 2 parts to about 16 parts by weight, and in some embodiments, about 4 parts to about 10 parts by weight, based on 100 parts by weight of the total weight of the composite formulation. The amount of the glidant may be in a range of about 0.1 parts to about 5 parts by weight, and in some embodiments, about 0.5 parts to about 2 parts by weight, based on 100 parts by weight of the total weight of the composite formulation.

For example, the diluent may be selected from the group consisting of lactose, calcium dihydrogen phosphate, starch, mannitol, microcrystalline cellulose, carboxy methyl cellulose, and any combination thereof, but is not limited thereto. For example, the binder may be selected from the group consisting povidone, hydroxypropyl cellulose, hydroxypropyl methylcellulose, pregelatinized starch, copovidone, and any combination thereof, but is not limited thereto. For example, the disintegrant may be selected from the group consisting of crospovidone, croscarmellose sodium, sodium starch glycolate, low-substituted hydroxypropyl cellulose, and any combination thereof, but is not limited thereto. For example, the glidant may be selected from the group consisting of stearic acid, metal salts of stearic acid, talc, colloidal silica, sucrose fatty acid esters, hydrogenated vegetable oils, wax, glyceryl fatty acid esters, and any combination thereof, but is not limited thereto

In some embodiments, the dissolution rates of the active ingredients may be improved by appropriately selecting specific additives (refer to Experimental Examples 2 and 4).

In some embodiments, the tadalafil wet granules-part may include a water-soluble diluent. The water-soluble diluent may be selected from the group consisting of lactose, mannitol, and any combination thereof. The water-soluble diluents may facilitate disintegration of granules in the tadalafil wet granules-part. The amount of the water-soluble diluent may be in a range of about 20 parts to about 60 parts by weight based on 100 parts by weight of the total weight of the composite formulation. When the amount of the water-soluble diluent in the tadalafil wet granules-part wet granules-part is within this range), the dissolution rate of tadalafil may be about 70% or higher in 15 minutes, which is higher than that of commercially available Cialis®. However, when the amount of the water-soluble diluent is less than 20 parts by weight, the dissolution rate of tadalafil may be markedly reduced to about 60% or less in 15 minutes, as experimentally supported in Experimental Example 2.

In some embodiments, the amlodipine mixture-part may include sodium starch glycolate as a disintegrant. Sodium starch glycolate may facilitate disintegration of tablets. The amount of the sodium starch glycolate may be in a range of about 2 parts to about 16 parts by weight, and in some embodiments, about 4 parts to about 10 parts by weight, based on 100 parts by weight of the total weight of the composite formulation. When the amount of the sodium starch glycolate in the amlodipine mixture-part is between about 4 parts to about 10 parts by weight, the dissolution rate of amlodipine may be about 70% or higher in 15 minutes, which is higher than that of commercially available Norvasc®. However, when the amount of the sodium starch glycolate is less than about 4 parts by weight, the dissolution rate of amlodipine may be markedly reduced to about 60% or less in 15 minutes. When the amount of the sodium starch glycolate is more than about 10 parts by weight, it may be difficult to maintain the appearance of tablets when exposed to accelerated conditions (refer to Experimental Example 4).

In some embodiments, the composite formulation may include: a tadalafil wet granules-part including tadalafil or a pharmaceutically acceptable salt thereof; and an amlodipine mixture-part including amlodipine or a pharmaceutically acceptable salt thereof, wherein the tadalafil wet granules-part may include about 20 parts to about 60 parts by weight of a water-soluble diluent selected from the group consisting of lactose, mannitol, and a combination thereof, based on 100 parts by weight of the total weight of the composite formulation, and the amlodipine mixture-part may include about 4 parts to about 10 parts by weight of sodium starch glycolate based on 100 parts by weight of the total weight of the composite formulation.

Non-limiting examples of the pharmaceutically acceptable salt of tadalafil are hydrobromide, phosphate, sulfate, hydrochloride, maleate, fumarate, lactate, tartrate, citrate, besylate, camsylate, and gluconate. In some embodiments, the tadalafil may be tadalafil in free form.

The composite formulation may include tadalafil in free form at about 5 mg to about 20 mg a unit dosage form, and in some embodiments, at about 5 mg to about 10mg a unit dosage form, in consideration of a known daily dose of tadalafil or a pharmaceutically acceptable salt thereof.

Examples of the pharmaceutically acceptable salt of amlodipine includes hydrobromide, phosphate, sulfate, hydrochloride, maleate, fumarate, lactate, tartrate, citrate, besylate, camsylate, and gluconate, but is not limited thereto. In one embodiment, the pharmaceutically acceptable salt of amlodipine may be camsylate or besylate.

The composite formulation may include amlodipine in free form at about 1.25 mg to about 20 mg a unit dosage form, and in some embodiments, at about 2.5 mg to about 10 mg a unit dosage form, and in some other embodiments, about 5 mg to about 10 mg a unit dosage form, in consideration of a known daily dose of amlodipine or a pharmaceutically acceptable salt thereof.

The composite formulation may be administered once a day, and daily.

In some embodiments, the composite formulation may be an oral formulation in the form of a tablet, a capsule, or multiple particles, but is not limited thereto. The tablet may be a single-layer tablet or a double-layer tablet. When the composite formulation is in capsule form, the tadalafil wet granules-part and the amlodipine mixture-part may be included in the capsule. The composite formulation may also be orally or sublingually administered. For example, the composite formulation may be orally administered.

The composite formulation including tadalafil or a pharmaceutically acceptable salt thereof, and amlodipine or a pharmaceutically acceptable salt thereof as active ingredients may be used for preventing or treating a cardiovascular disease, an erectile dysfunction, or a combination thereof. For example, the composite formulation may be useful for a patient with hypertension optionally accompanied by an erectile dysfunction due to a vasodilation effect of tadalafil as a PDE-5 inhibitor and an antihypertensive function of amlodipine used as a calcium channel antagonist.

The cardiovascular disease may be any cardiovascular disease known as an indication of an amlodipine. For example, the cardiovascular disease may be selected from the group consisting of angina pectoris, hypertension, artery spasm, cardiac arrhythmias, cardiac hypertrophy, stroke, congestive heart failure, and myocardial infarction. In particular, since daily dose of both tadalafil and amlodipine are included in a single formulation, the composite formulation may prevent or treat both a cardiovascular disease and erectile dysfunction through once-daily dosing and thus may markedly improve a patient's compliance in a patient with both a cardiovascular disease and an erectile dysfunction or with the risk of the two diseases.

According to another aspect of the present disclosure, a method of preparing any of the composite formulations according to the above-described embodiments includes:

preparing a tadalafil wet granules-part including tadalafil or a pharmaceutically acceptable salt thereof, and a pharmaceutically acceptable additive;

preparing an amlodipine mixture-part including amlodipine or a pharmaceutically acceptable salt thereof, and a pharmaceutically acceptable additive; and

formulating the tadalafil wet granules-part and the amlodipine mixture-part together.

The detailed descriptions of any of the composite formulations according to the above-described embodiments may apply to the embodiments of methods of preparing any of the composite formulations.

The tadalafil wet granules-part may be prepared by wet granulation. The wet granulation is not specifically limited, and may be any wet granulation known in the art.

In the method of preparing a composite formulation, an embodiment of preparing of the wet granules-part may include:

(a) blending tadalafil or a pharmaceutically acceptable salt thereof with a water-soluble diluent;

(b) adding a binder solution to a mixture of step (a) while blending the mixture;

(c) drying a resultant of step (b); and

(d) grinding or sieving a resultant of step (c).

The water-soluble diluent in step (a) may be selected from the group consisting of mannitol, lactose, and a combination thereof.

A solvent for preparing the binder solution of step (b) may be water, ethanol, isopropanol, acetone, or any combination thereof. The binder solution may be prepared by adding any additive available in the pharmaceutical field, for example, by adding a binder, a surfactant, a buffer, or a combination thereof. In some embodiments, the water-soluble diluent may include mannitol, and the binder solution may include sodium lauryl sulfate, hydroxypropyl cellulose, and pregelatinized starch.

The drying in step (c) may be performed at a temperature not higher than about 60□, and in some embodiments, not higher than about 50° C., and in some other embodiments, not higher than about 40° C., in consideration of stability of the active ingredients. For example, the drying in step (c) may be performed at a temperature of about 20° C. to about 40° C. by air drying, fluid-bed drying, oven drying, or microwave drying.

The grinding or sieving in step (d) may be performed using a sieve with a mesh size of about 20 to about 30.

In the method of preparing a composite formulation according to any of the above-described embodiments, the preparing of the amlodipine mixture-part may include directly adding amlodipine or a pharmaceutically acceptable salt thereof, and a pharmaceutically acceptable additive to the tadalafil wet granules-part and mixing together.

In the preparing of the amlodipine mixture-part, the pharmaceutically acceptable additive may be appropriately selected from a disintegrant, a diluent, a glidant, and any combination thereof. In some embodiments, the pharmaceutically acceptable additive may include sodium starch glycolate, a diluent, and a glidant. In some embodiments, the diluent may include microcrystalline cellulose, mannitol, or a combination thereof. For example, the glidant may include magnesium stearate.

The mixing may be performed using any mixer commonly used in the art, for example, a tumble bin, a V-mixer, or any appropriate mixer.

In the method of preparing a composite formulation according to any of the above-described embodiments, the formulating may be formulating the tadalafil wet granules-part and the amlodipine mixture-part together as a capsule, a tablet, or multiple particles. In some embodiments, the formulating may be tableting. For tableting a mixture of the tadalafil wet granules-part and the amlodipine mixture-part, any tablet presses conventionally used in the art, for example, a rotary tablet press may be used.

In an embodiment of the method of preparing a composite formulation according to any of the above-described embodiments, the pharmaceutically acceptable additive of the tadalafil wet granules-part includes sodium lauryl sulfate, hydroxypropyl cellulose, pregelatinized starch, and mannitol, the pharmaceutically acceptable additive of the amlodipine mixture-part includes sodium starch glycolate, a diluent, and a glidant, and the formulating is tableting.

In some embodiments, the method of preparing a composite formulation according to any of the above-described embodiments may further include film-coating a tablet resulting from the tableting.

A polymer film coating layer formed through the film-coating may provide the tablet with an appropriate hardness of about 5 kp to about 20 kp, and in some embodiments, about 6 kp to about 13 kp. The polymer of the polymer film coating layer may be any conventional pharmaceutically acceptable polymer which is able to form a film coating. For example, the amount of the polymer may be in a range of about 1 part to about 10 parts by weight, and in some embodiments, about 3 parts to about 5 parts by weight, based on 100 parts by weight of the total weight of the composite formulation.

In some embodiments, a method of preparing a composite formulation according to any of the above-described embodiments may include: (i) preparing a tadalafil wet granules-part including tadalafil or a pharmaceutically acceptable salt thereof, and a pharmaceutically acceptable additive; (ii) preparing an amlodipine mixture-part including amlodipine or a pharmaceutically acceptable salt thereof, and a pharmaceutically acceptable additive; and (iii) mixing the tadalafil wet granules-part of step (i) and the amlodipine mixture-part of step (ii), and preparing a single-layered tablet using a common tablet preparation method.

In some other embodiments, a method of preparing a composite formulation according to any of the above-described embodiments may include: (i) preparing a tadalafil wet granules-part including tadalafil or a pharmaceutically acceptable salt thereof, and a pharmaceutically acceptable additive; (ii) preparing an amlodipine mixture-part including amlodipine or a pharmaceutically acceptable salt thereof, and a pharmaceutically acceptable additive; and (iii) preparing a double-layered tablet including a first layer of the tadalafil wet granules-part of step (i) and a second layer of the amlodipine mixture-part of step (ii) by using a common double-layer tablet preparation method.

In some other embodiments, a method of preparing a composite formulation according to any of the above-described embodiments may include: (i) preparing a tadalafil wet granules-part including tadalafil or a pharmaceutically acceptable salt thereof, and a pharmaceutically acceptable additive; (ii) preparing an amlodipine mixture-part including amlodipine or a pharmaceutically acceptable salt thereof, and a pharmaceutically acceptable additive; and (iii) filling a capsule with a mixture of the tadalafil wet granules-part of step (i) and the amlodipine mixture-part of step (ii) to formulate the composite formulation as a capsule.

Thereinafter, one or more embodiments of the present invention will be described in detail with reference to the following examples. However, these examples are not intended to limit the scope of the one or more embodiments of the present invention.

### EXAMPLES 1 to 6

Composite formulations in solid form of Examples 1 to 6 were prepared according to the compositions represented in Table 1.

Tablets of the composite formulations were prepared by wet granulation. First, tadalafil was mixed with mannitol as a water-soluble diluent, pregelatinized starch, and hydroxypropyl cellulose. The resulting mixture in powder form was wet-granulated using a binder solution including hydroxypropyl cellulose and sodium lauryl sulfate in water. The resulting tadalafil wet granules mixture was dried using a fluid-bed drying or a dry oven, followed by adding amlodipine besylate, microcrystalline cellulose, mannitol, and sodium starch glycolate thereto and mixing with a V-mixer at about 40 rpm for about 30 minutes (Primary mixing). The resulting primary-mixing product was mixed with magnesium stearate at about 40 rpm for about 5 minutes (Secondary mixing). The resulting mixture was tableted using a tablet press.

TABLE 1

| | | Example | Example | Example | Example | Example | Example |
|---|---|---|---|---|---|---|---|
| Ingredients | | 1 | 2 | 3 | 4 | 5 | 6 |
| Wet | tadalafil | 5.0 | 5.0 | 5.0 | 5.0 | 5.0 | 5.0 |
| granulation | mannitol | 30.0 | 15.0 | 77.0 | 30.0 | 30.0 | 30.0 |
| | pregelatinized starch | 4.0 | 4.0 | 4.0 | — | 4.0 | 4.0 |
| | hydroxypropyl cellulose | 3.4 | 3.4 | 3.4 | 3.4 | 3.4 | 3.4 |
| Binder | hydroxypropyl cellulose | 0.6 | 0.6 | 0.6 | 0.6 | 0.6 | 0.6 |
| solution | sodium lauryl sulfate | 0.6 | 0.6 | 0.6 | 0.6 | 0.6 | 0.6 |
| Primary | Amlodipine besylate | 6.94 | 6.94 | 6.94 | 6.94 | 6.94 | 13.88 |
| mixing | (as amlodipine) | (5.0) | (5.0) | (5.0) | (5.0) | (5.0) | (10.0) |
| | microcrystalline cellulose | 25.96 | 25.96 | 25.96 | 25.96 | 25.96 | 25.96 |
| | mannitol | 47.0 | — | — | 45.0 | 41.0 | 40.06 |
| | sodium starch glycolate | 6.0 | 3.0 | 6.0 | 12.0 | 12.0 | 6.0 |
| Secondary | magnesium stearate | 1.5 | 1.5 | 1.5 | 1.5 | 1.5 | 1.5 |
| mixing | | | | | | | |

| | | | | | | |
|---|---|---|---|---|---|---|
| Total weight (mg) | 131.0 | 66.0 | 131.0 | 131.0 | 131.0 | 131.0 |

### Preparation Examples 1 to 3

Composite formulations in solid form of Preparation Examples 1 to 3 were prepared in the same manner as in Example 1, except that amlodipine was used together with tadalafil during the wet granulation, as represented in Table 2.

TABLE 2

| | | Prepa- | Prepa- | Prepa- |
|---|---|---|---|---|
| | | ration | ration | ration |
| Ingredients | | Example 1 | Example 2 | Example 3 |
| Wet | tadalafil | 5.0 | 5.0 | 5.0 |
| granulation | amlodipine besylate | 6.94 | 6.94 | 6.94 |
| | (as amlodipine) | (5.0) | (5.0) | (5.0) |
| | mannitol | 0 | 15.0 | 30.0 |
| | pregelatinized | 4.0 | 4.0 | 4.0 |
| | starch | | | |
| | hydroxypropyl | 3.4 | 3.4 | 3.4 |
| | cellulose | | | |
| Binder | hydroxypropyl | 0.6 | 0.6 | 0.6 |
| solution | cellulose | | | |
| | sodium lauryl | 0.6 | 0.6 | 0.6 |
| | sulfate | | | |
| Primary | microcrystalline | 25.96 | 25.96 | 25.96 |
| mixing | cellulose | | | |
| | mannitol | 77.0 | 62.0 | 47.0 |
| | sodium starch | 6.0 | 6.0 | 6.0 |
| | glycolate | | | |
| Secondary | magnesium | 1.5 | 1.5 | 1.5 |
| mixing | stearate | | | |

| | | | |
|---|---|---|---|
| Total weight (mg) | 131.0 | 131.0 | 131.0 |

### Preparation Examples 4 and 5, and Comparative Example 1

Composite formulations in solid form of Preparation Examples 4 to 5 and Comparative Example 1 were prepared according to the compositions represented in Table 3.

TABLE 3

| | | Prepa- | Prepa- | Com- |
|---|---|---|---|---|
| | | ration | ration | parative |
| Ingredients | | Example 4 | Example 5 | Example 1 |
| Wet | tadalafil | 5.0 | 5.0 | 5.0 |
| granulation | mannitol | 30.0 | 30.0 | 90.0 |
| | pregelatinized starch | 4.0 | 4.0 | 4.0 |
| | hydroxypropyl | 3.4 | 3.4 | 3.4 |
| | cellulose | | | |
| Binder | hydroxypropyl | 0.6 | 0.6 | 0.6 |
| solution | cellulose | | | |
| | sodium lauryl sulfate | 0.6 | 0.6 | 0.6 |
| Primary | amlodipine | 6.94 | 6.94 | 6.94 |
| mixing | besylate | | | |
| | (as amlodipine) | (5.0) | (5.0) | (5.0) |
| | microcrystalline | 25.96 | 25.96 | 25.96 |
| | cellulose | | | |
| | mannitol | 53.0 | 35.0 | 219.0 |
| | sodium starch | 0 | 18.0 | 18.0 |
| | glycolate | | | |
| Secondary | magnesium stearate | 1.5 | 1.5 | 1.5 |
| mixing | | | | |

| | | | |
|---|---|---|---|
| Total weight (mg) | 131.0 | 131.0 | 375.0 |

Relative proportions (weight %) of the ingredients in the composite formulations of Examples 1 to 6, Preparation Examples 1 to 5, and Comparative Example 1 based on a total weight of the composite formulation are shown in Table 4.

TABLE 4

| | Main ingredient | | |
|---|---|---|---|
| | (Total of two active | Mannitol of wet | Sodium starch |
| Example | ingredients) (%) | granules-part (%) | glycolate (%) |
| Example1 | 7.6% | 22.9% | 4.6% |
| Example2 | 15.2% | 22.7% | 4.5% |
| Example3 | 7.6% | 58.8% | 4.6% |
| Example4 | 7.6% | 22.9% | 4.6% |
| Example5 | 7.6% | 22.9% | 9.2% |
| Example6 | 11.5% | 22.9% | 4.6% |
| Preparation | 7.6% | 0% | 4.6% |
| Example 1 | | | |
| Preparation | 7.6% | 11.5% | 4.6% |
| Example 2 | | | |
| Preparation | 7.6% | 22.9% | 4.6% |
| Example 3 | | | |
| Preparation | 7.6% | 22.9% | 0% |
| Example 4 | | | |
| Preparation | 7.6% | 22.9% | 13.7% |
| Example 5 | | | |
| Comparative | 2.7% | 24.0% | 4.8% |
| Example 1 | | | |

### Experimental Example 1

### Tablet Size Comparison

Tablet sizes of Examples 1 and 2 and Comparative Example 1, and Norvasc® and Cialis® as commercially available products were measured using vernier calipers. Weights thereof were also measured using a scale. The results are shown in Table 5.

TABLE 5

| | | | Com- | | |
|---|---|---|---|---|---|
| Size | | | parative | | |
| and mass | Example1 | Example2 | Example 1 | Norvasc ® | Cialis ® |
| Main axis | 9.58 | 5.55 | 13.66 | 8.74 | 9.78 |
| (mm) | | | | | |
| Minor axis | 4.55 | 5.55 | 6.64 | 6.23 | 6.03 |
| (mm) | | | | | |
| Weight | 131 | 66 | 375 | 200 | 175 |
| (mg) | | | | | |

Referring to Table 5 and FIG. 1, the tablet size of the composite formulation of Comparative Example 1 was found to be markedly larger than the other tablets. Accordingly, although taking only a single tablet of the composite formulation of Comparative Example 1, is convenient for a patient, the composite formulation of Comparative Example 1 having such a large tablet size may not be easy to swallow, which does not significantly increase a patient's compliance, compared to taking commercially available single formulations twice.

However, the tablets of the composite formulations of Examples 1 and 2, including both the two active ingredients tadalafil and amlodipine, were found to have smaller sizes and smaller weight than each of the commercially available single formulations. Therefore, the composite formulations of Examples 1 and 2 were found to be convenient to swallow even though they include both the two active ingredients tadalafil and amlodipine in a single tablet, and thus the composite formulations of Examples 1 and 2 have significantly improved patient compliance.

### Experimental Example 2

### Tadalafil Dissolution Rate With Respect to the Percentage of Water-Soluble Diluent Tadalafil Wet Granules-Part

Tadalafil dissolution rates of the composite formulations of Examples 1, 3, and 4, and Preparation Examples 1 and 2 were evaluated according to the USP37 dissolution test method for tadalafil tablets under the following conditions.

<Dissolution Conditions>

Dissolution medium: 0.5% sodium lauryl sulfate solution, 1000 mL

Apparatus: Apparatus II (paddle method), 50 rpm

Temperature: 37□

<Analysis Conditions>

Column: Stainless column having an inner diameter of about 4.6 mm and a length of about 15 cm, filled with octadecylsilyl-silica gel for liquid chromatography (LC) having a particle diameter of about 3.5 μm

Mobile phase: Mixed solution of methanol and purified water in 50:50

Detector: Ultraviolet (UV)-absorption photometer (measurement wavelength: 225 nm)

Flow rate: 2.0 mL/min

Injection volume: 50 μl

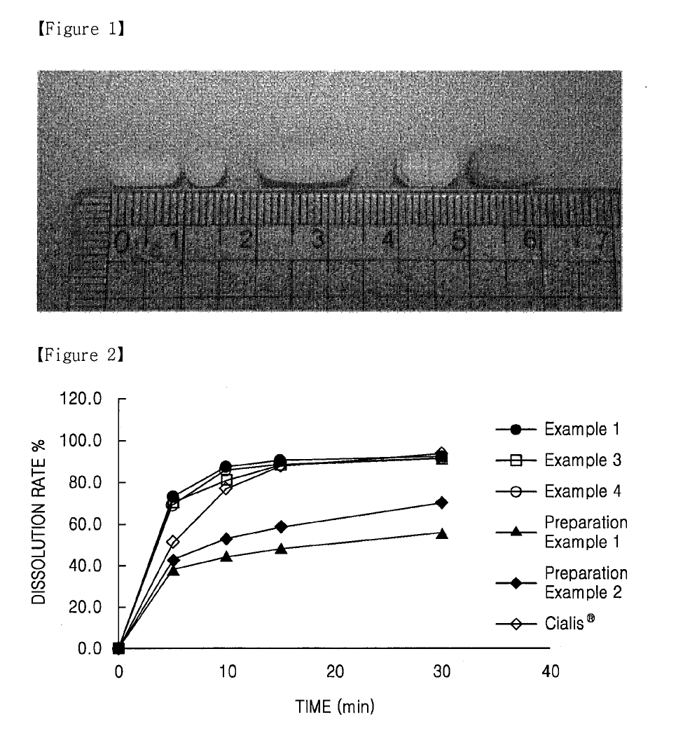

The resulting tadalafil dissolution rates of the composite formulations of Examples 1, 3, and 4, Preparation Examples 1 and 2, and Cialis® are shown in Table 6 and FIG. 2.

TABLE 6

Dissolution rate (%)

| Example | 5 min | 10 min | 15 min | 30 min |
|---|---|---|---|---|
| Example 1 | 73.2 | 87.1 | 90.6 | 93.0 |
| Example 3 | 70.5 | 81.4 | 88.6 | 91.2 |
| Example 4 | 68.5 | 85.3 | 88.2 | 91.3 |
| Preparation Example 1 | 38.5 | 44.5 | 48.5 | 55.7 |
| Preparation Example 2 | 53.1 | 64.5 | 70.1 | 75.9 |
| Cialis ® | 51.1 | 77.2 | 87.9 | 94.0 |

Referring to Table 6 and FIG. 2, the composite formulations of Examples 1, 3, and 4 were found to have a markedly high tadalafil dissolution rate of about 70% or more in 15 minutes, while the composite formulations of Preparation Examples 1 and 2 were found to have a low tadalafil dissolution rate of less than about 70% in 15 minutes.

These results indicate that the tadalafil dissolution rate of a composite formulation including tadalafil and amlodipine may be effectively improved when the amount of mannitol, a water-soluble diluent in the wet granules-part of the composite formulation is in a range of about 20 wt% to about 60 wt%, compared to when it is in a range of about 0 wt% to about 12 wt%.

### Experimental Example 3

### Comparison of Amlodipine Dissolution Rate With Respect to Method of Adding Amlodipine

Amlodipine dissolution rates of the composite formulations of Example 1 and

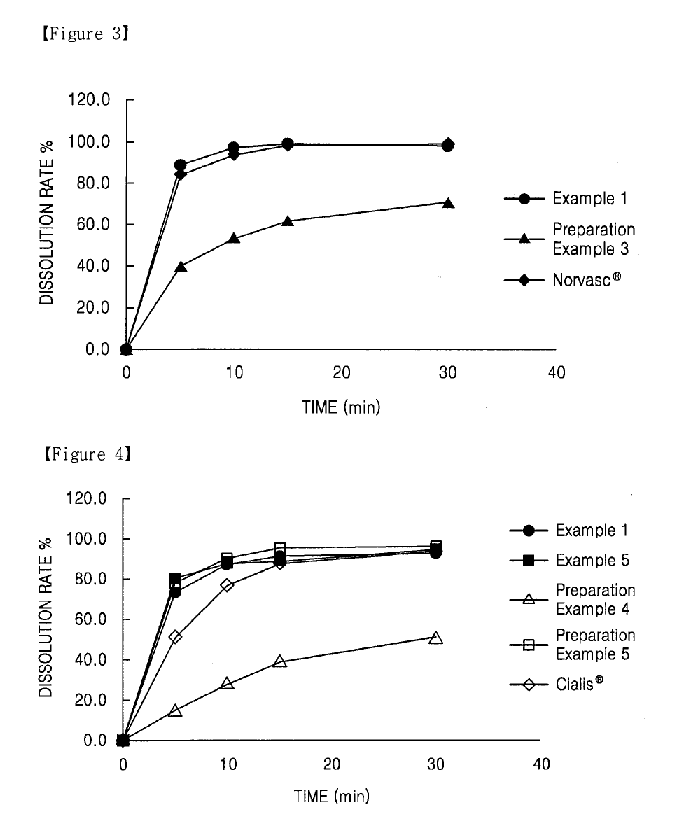

Preparation Example 3 were evaluated according to the USP37 dissolution test method for amlodipine besylate tablets. The results are shown in Table 7 and FIG. 3.

TABLE 7

Dissolution rate (%)

| Example | 5 min | 10 min | 15 min | 30 min |
|---|---|---|---|---|
| Example 1 | 88.4 | 96.8 | 98.8 | 98.4 |
| Preparation Example 3 | 40.1 | 53.7 | 61.8 | 71.1 |
| Norvasc ® | 84.5 | 94.3 | 98.7 | 99.5 |

Referring to Table 7 and FIG. 3, the composite formulations of Example 1 and the commercially available Norvasc® were found to have a markedly high amlodipine dissolution rate of about 70% or more in 15 minutes, while the composite formulation of Preparation Example 3 was found to have a low amlodipine dissolution rate of about 60% or less in 15 minutes.

These results indicate that the amlodipine dissolution rate of a composite formulation including amlodipine in a post-mixture-part separated from the tadalafil wet granules-part may be high, similar to that of the commercially available Norvasc®, since amlodipine can dissolve fast at the same time of tablet disintegration. Meanwhile, a composition formulation including both amlodipine and tadalafil in a wet granules-part as in the composite formulation of Preparation Example 3 may delay dissolution of amlodipine. The results indicate that including amlodipine in a post-mixture-part separated from the tadalafil wet granules-part may be better in terms of amlodipine dissolution rate.

### Experimental Example 4

### Comparison of Tadalafil Dissolution Rate With Respect to the Amount of Disinteqrant, and Tablet's Appearance in Accelerated Exposure Conditions

Tadalafil dissolution rates of the composite formulations of Examples 1 and 5 and Preparation Examples 4 and 5 were evaluated in the same manner as in Experimental Example 2. The results are shown in Table 8 and FIG. 4.

After exposure to accelerated conditions (40° C., 75% relative humidity (RH)), changes in tablet thickness and hardness with respect to time were evaluated. The results are shown in Tables 9 and 10. The evaluation of changes in such appearance may help to predict the appearance stabilities of a composite formulation when stored under high temperature and high humidity conditions as in the summer.

TABLE 8

Dissolution rate (%)

| Example | 5 min | 10 min | 15 min | 30 min |
|---|---|---|---|---|
| Example 1 | 73.2 | 87.1 | 90.6 | 93.0 |
| Example 5 | 80.4 | 87.6 | 88.9 | 94.2 |
| Preparation Example 4 | 15.3 | 28.4 | 39.4 | 51.2 |
| Preparation Example 5 | 78.6 | 90.1 | 93.3 | 94.1 |

TABLE 9

Tablet thickness (mm)

| Example | 0 h | 6 h | 12 h | 24 h |
|---|---|---|---|---|
| Example 1 | 3.32 | 3.38 | 3.38 | 3.38 |
| Example 5 | 3.33 | 3.35 | 3.38 | 3.39 |
| Preparation Example 4 | 3.32 | 3.37 | 3.37 | 3.38 |
| Preparation Example 5 | 3.32 | 3.69 | 3.69 | 3.70 |

TABLE 10

Hardness(kp)

| Example | 0 h | 6 h | 12 h | 24 h |
|---|---|---|---|---|
| Example 1 | 8.5 | 7.2 | 7.0 | 6.9 |
| Example 5 | 8.1 | 7.4 | 6.8 | 6.6 |
| Preparation Example 4 | 8.9 | 7.4 | 7.2 | 6.8 |
| Preparation Example 5 | 8.4 | 6.1 | 5.2 | 4.4 |

Referring to Tables 8, 9, and 10, when a disintegrant is used in an amount of about 4% to about 10% based on the tablet weight, the tablet shows better results in terms of dissolution rate and appearance. However, when a disintegrant is used in an amount of less than 4% based on the tablet weight shows a low dissolution rate that is different from the dissolution profiles of a commercially available product, and thus was considered not to have the same efficacy as a commercially available product. In addition, when a disintegrant is used in an amount of more than 10% based on the tablet weight , the tablet is shown to swell rapidly and to have a decreasing hardness with time, thus having poor stability in appearance during storage. Therefore, a composite formulation according to any of the above-described embodiments may include about 4 parts to about 10 parts by weight of sodium starch glycolate with respect to 100 parts by weight of a total weight of the composite formulation.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

1. A composite formulation comprising:
tadalafil or a pharmaceutically acceptable salt thereof and amlodipine or a pharmaceutically acceptable salt thereof as active ingredients,
wherein a total amount of tadalafil and amlodipine is in a range of about 6 parts to about 16 parts by weight based on 100 parts by weight of a total weight of the composite formulation.

2. The composite formulation of claim 1, wherein tadalafil and amlodipine in the composite formulation each have a dissolution rate of about 70% or more in 15 minutes, as tested by the United States Pharmacopeia (USP) 37 dissolution test.

3. The composite formulation of claim 1, wherein the composite formulation comprises:
a tadalafil wet granules-part comprising tadalafil or a pharmaceutically acceptable salt thereof; and
an amlodipine mixture-part comprising amlodipine or a pharmaceutically acceptable salt thereof.

4. The composite formulation of claim 3, wherein the tadalafil wet granules-part or the amlodipine mixture-part comprises at least one pharmaceutically acceptable additive selected from the group consisting of a diluent, a disintegrant, a binder, and a glidant.

5. The composite formulation of claim 4, wherein the tadalafil wet granules-part comprises a water-soluble diluent selected from the group consisting of lactose, mannitol, and a combination thereof.

6. The composite formulation of claim 5, wherein an amount of the water-soluble diluent is in a range of about 20 parts to about 60 parts by weight based on 100 parts by weight of the total weight of the composite formulation.

7. The composite formulation of claim 4, wherein the amlodipine mixture-part comprises sodium starch glycolate as a disintegrant.

8. The composite formulation of claim 7, wherein the amlodipine mixture-part comprises about 4 parts to about 10 parts by weight of sodium starch glycolate based on 100 parts by weight of the total weight of the composite formulation.

9. The composite formulation of claim 4, wherein the tadalafil wet granules-part comprises about 20 parts to about 60 parts by weight of a water-soluble diluent selected from the group consisting of lactose, mannitol, and a combination thereof, based on 100 parts by weight of the total weight of the composite formulation, and
the amlodipine mixture-part comprises about 4 parts to about 10 parts by weight of sodium starch glycolate based on 100 parts by weight of the total weight of the composite formulation.

10. The composite formulation of claim 1, wherein an amount of tadalafil is in a range of about 5 mg to about 10 mg a unit dosage form.

11. The composite formulation of claim 1, wherein an amount of amlodipine is in a range of about 5 mg to about 10 mg a unit dosage form.

12. The composite formulation of claim 1, wherein the composite formulation is administered once a day.

13. The composite formulation of claim 1, wherein the composite formulation is administered daily.

14. The composite formulation of claim 1, wherein the composite formulation is an oral formulation in the form of a tablet, a capsule, or multiple particles.

15. The composite formulation of claim 1, wherein the composite formulation is a preventive or therapeutic formulation for a cardiovascular disease, an erectile dysfunction, or a combination thereof.

16. A method of preparing the composite formulation of any claim 1, the method comprising:
preparing a tadalafil wet granules-part comprising tadalafil or a pharmaceutically acceptable salt thereof, and a pharmaceutically acceptable additive;
preparing an amlodipine mixture-part comprising amlodipine or a pharmaceutically acceptable salt thereof, and a pharmaceutically acceptable additive; and
formulating the tadalafil wet granules-part and the amlodipine mixture-part together.

17. The method of claim 16, wherein the pharmaceutically acceptable additive of the tadalafil wet granules-part comprises sodium lauryl sulfate, hydroxypropyl cellulose, pregelatinized starch, and mannitol,
the pharmaceutically acceptable additive of the amlodipine mixture-part comprises sodium starch glycolate , a diluents, and a glidant, and the formulating comprises tableting.

18. The method of claim 17, wherein the diluents is microcrystalline cellulose, mannitol, or a combination thereof, and the glidant is magnesium stearate.

19. A method of preparing the composite formulation of claim 15, the method comprising:
preparing a tadalafil wet granules-part comprising tadalafil or a pharmaceutically acceptable salt thereof, and a pharmaceutically acceptable additive;
preparing an amlodipine mixture-part comprising amlodipine or a pharmaceutically acceptable salt thereof, and a pharmaceutically acceptable additive; and
formulating the tadalafil wet granules-part and the amlodipine mixture-part together.
